SEQUENCE LISTING (2 sequences)

SEQ ID NO: 1 - protein, 126 aa, Homo sapiens
EVQLVQSGAEVKKPGSSVKVSCKASGGTFSSYAISWVRQAPGQGLEWMGGIIPIFGTANYAQKFQGRVTITADESTSTAYMELSSLRSEDTAVYYCAREEGPYCSSTSCYGAFDIWGQGTLVTVSS

SEQ ID NO: 2 - protein, 108 aa, Homo sapiens
QSVLTQDPAVSVALGQTVKITCQGDSLRSYFASWYQQKPGQAPTLVMYARNDRPAGVPDRFSGSKSGTSASLAISGLQSEDEADYYCAAWDDSLNGYLFGAGTKLTVL

(19) United States
(12) Patent Application Publication
(10) Pub. No.: US 2013/0259944 A1
(43) Pub. Date: Oct. 3, 2013

(54) METHODS AND COMPOSITIONS FOR TREATING CANCER WITH PLATINUM PARTICLES

(71) Applicants: Dong M. Shin, Atlanta, GA (US); Shuming Nie, Atlanta, GA (US); XiangHong Peng, Atlanta, GA (US); Yiqing Wang, Atlanta, GA (US)

(72) Inventors: Dong M. Shin, Atlanta, GA (US); Shuming Nie, Atlanta, GA (US); XiangHong Peng, Atlanta, GA (US); Yiqing Wang, Atlanta, GA (US)

(73) Assignee: EMORY UNIVERSITY, Atlanta, GA (US)

(21) Appl. No.: 13/791,027

(22) Filed: Mar. 8, 2013

Related U.S. Application Data
(60) Provisional application No. 61/619,511, filed on Apr. 3, 2012.

(51) Int. Cl. A61K 9/14

(52) U.S. Cl. CPC A61K 9/14
USPC 424/491; 536/21; 424/649

(57) ABSTRACT

This disclosure relates to methods and compositions for treating cancer with platinum particles. In certain embodiments, the disclosure relates to platinum particle coated with a polysaccharide, such as a heparin or modified heparin, conjugated to a polypeptide that has affinity for a cell surface cancer marker and uses related thereto.

### CROSS REFERENCE TO RELATED APPLICATIONS

This application claims priority to U.S. provisional application No. 61/619,511 filed Apr. 3, 2012, hereby incorporated by reference in its entirety.

### STATEMENT REGARDING FEDERALLY FUNDED RESEARCH

This invention was made with government support under grant U54CA119338, U01 CA151802, and P50CA128613 from the National Institute of Health. The government has certain rights in the invention.

### BACKGROUND

The anticancer drug cis-diamminedichloroplatinum(II) (cisplatin, DDP) has been used in the treatment of different kinds of solid tumors including head and neck squamous cell carcinoma, ovarian, and non-small cell lung cancers (NSCLC). However, the more extensive use of cisplatin is limited by its severe side effects, such as nephrotoxicity and peripheral neuropathy, which may result from its nonspecific systemic organ distribution and inadequate intratumor concentrations.

Nanoparticles have been reported improve drug specificity. Non-targeted nanoparticles are usually absent in the tumor sites due to their lack of cellular uptake, while the tumor-targeted nanoparticles can enter tumor cells via receptor-mediated internalization. See Bae, J. Controlled Release, 2009, 133, 2-3 and Allen & Cullis, Science, 2004, 303, 1818-1822. A variety of tumor targeting ligands, such as antibodies, peptides, and small molecules, have been used to facilitate the uptake of nanoparticles into target cells. Overexpression of the epidermal growth factor receptor (EGFR) has been found in about 40-80% of NSCLC tissues, making it an ideal tumor target for constructing nanoparticles for targeted NSCLC therapy. Although an EGFR monoclonal antibody has been widely used to engineer tumor-targeted nanoparticles for either tumor imaging or selective drug delivery, the high molecular weight of the full-length antibody may limit its penetration into tumor tissue, and the specificity of tumor-targeted nanoparticles may be affected by the interaction of whole antibody with Fc receptors on normal tissues. Single-chain antibodies against EGFR (ScFvEGFR, MW 25 kDa), which contain the specific EGFR binding region but lack the Fc region, have been isolated and their specificity of binding and internalization demonstrated. See Yang et al., Small, 2009, 5, 235-243: Horak et al., Cancer Biother. Radiopharm., 2005, 20, 603-613; and Zhou et al., J. Mol. Biol. 2007, 371, 934-947.

Nishiyama et al. report cisplatin-incorporated polymeric micelles can eradicate solid tumors in mice. See Cancer Res, 2003, 63, 8977-8983. Jeong et al. report cisplatin-incorporated hyaluronic acid nanoparticles based on ion-complex formation. See J Pharm Sci, 2008, 97, 1268-1276. See also Gryparis et al., Eur J Pharm Biopharm, 2007, 67, 1-8 and Kim et al., J Controlled Release, 2008, 127, 41-49. These delivery systems only allow a proportion of the drug-loaded nanoparticles to enter the tumor cells due to their lack of targeting. Thus, there is a need for improvements.

Wang et al. report a targeting nanoparticle that enhances specific delivery of paclitaxel to folate receptor-positive tumors. See ACS Nano, 2009, 3, 3165-3174. Tseng et al. report the use of biotinylated-EGF-modified gelatin nanoparticle carrier to enhance cisplatin accumulation in cancerous lungs via inhalation. See Biomaterials, 2009, 30, 3476-3485.

References cited herein are not an admission of prior art.

### SUMMARY

This disclosure relates to methods and compositions for treating cancer with platinum particles. In certain embodiments, the disclosure relates to platinum particles coated with a polysaccharide, such as a modified heparin, conjugated to a polypeptide that has affinity for a cell surface cancer marker and uses related thereto.

In certain embodiments, the disclosure relates to platinum complexes comprising at least one polysaccharide ligand such as heparin or modified heparin. In certain embodiments, the platinum complex is a platinum(II) complex and the polysaccharide ligand is heparin or modified heparin. In certain embodiments, the disclosure relates to platinum particles coated with a polysaccharide conjugated to a polypeptide that has affinity for a cell surface cancer marker wherein the polysaccharide is a heparin and the heparin is modified with succinic anhydride.

In certain embodiments, the heparin is conjugated to the polypeptide that targets EGFR such as the polypeptide ScFvEGFR.

In certain embodiments, the polypeptide that targets EGFR comprises SEQ ID NO: 1 or 2 or a polypeptide having 50%, 60%, 70%, 80%, 90%, 95% sequence identity or similarity thereto or having one, two, three, four, five, six, seven or eight mutations, additions, deletions, substitutions, or conserved substitutions therein.

In certain embodiments, the disclosure relates to pharmaceutical compositions comprising particles disclosed herein and a pharmaceutically acceptable excipient.

In certain embodiments, the disclosure relates to methods of treating cancer comprising administering an effective amount of a platinum particle disclosed herein to a subject in need thereof. In certain embodiments, the cancer is selected from head and neck squamous cell carcinoma, ovarian, and non-small cell lung cancers. In certain embodiments, the platinum particle is administered in combination with a second anti-cancer agent.

In certain embodiments, the disclosure relates to methods of preparing compositions disclosed herein by processes disclosed herein.

### BRIEF DESCRIPTION OF THE FIGURES

FIG. 1 illustrated the preparation of HDDP and EHDDP nanoparticles. (A) Schematic representation of HDDP and EHDDP nanoparticle formulations. (B) In vitro Pt release from EHDDP nanoparticles. The Pt loaded in the EHDDP nanoparticles shows sustained release in PBS (pH=7.4) at 37° C., while the nanoparticles are relatively stable in distilled water.

FIG. 2 shows data on intracellular Pt accumulation, cytotoxicity, and Pt-DNA adducts in vitro. (A) Accumulation of Pt in H292 (EGFR-positive) and H520 (EGFR-negative) NSCLC cells treated with DDP, HDDP, and EHDDP nanoparticles as described in Materials and Methods. (B) Effect of knockdown of EGFR on the internalization of EHDDP nanoparticles in NCI-H292 cells. (C) Percentage of viable cells measured by SRB assay in H292 cells treated with different concentrations of DDP, HDDP, and EHDDP nanoparticles for 96 h on 96-well plates, as described in Materials and methods. (D) Quantification of Pt in Pt-DNA adducts and expression of phosphorylation of H2A.X in H292 cells treated with DDP, HDDP, and EHDDP nanoparticles as described in Materials and Methods. The data are shown as mean±SD (n=3), *P<0.05, **P<0.01.

FIG. 3 shows data on the biodistribution of Pt in mice treated with DDP, HDDP, and EHDDP nanoparticles. Pt concentrations are shown in peripheral blood (A), liver, spleen, kidney, and tumor (B) of nude mice bearing H292 tumors at different time points after iv injection of a single dose of DDP, HDDP, and EHDDP nanoparticles (Pt 2.5 mg/kg, 3 mice per group). The data are shown as mean±SD, *P<0.05, **P<0.01.

FIG. 4 shows data on the Pt-DNA adducts in kidney and tumor. Pt concentration in Pt-DNA adducts in the kidney and tumor of nude mice bearing H292 tumors at 4 and 24 h after iv injection of a single dose of DDP, HDDP, and EHDDP nanoparticles (Pt 2.5 mg/kg, 3 mice per group). The data are shown as mean±SD, *P<0.05, **P<0.01.

FIG. 5 shows data on the effects of DDP, HDDP, and EHDDP nanoparticles on tumor growth and body weight. Tumor growth rate (A) and body weight change (%) (B) of nude mice treated with DDP, HDDP, and EHDDP nanoparticles (Pt 2.5 mg/kg, 5 injections, 3 day intervals, 6 mice per group). The data are shown as mean±SD, *P<0.05, **P<0.01.

FIG. 6 shows data on the comparison of side effects of DDP, HDDP, and EHDDP nanoparticles in the spleen. The weight change (A), actual size and shape of the spleen (B), and the histopathological results in the spleen 21 days after treatment with saline (C), DDP (D), HDDP (E), and EHDDP (F) nanoparticles (Pt 2.5 mg/kg, 5 injections, 3 day intervals, 6 mice per group), original magnification, ×200. The data are shown as mean±SD, **P<0.01.

FIG. 7 shows data on the comparison of side effects of DDP, HDDP, and EHDDP nanoparticles in the kidney. Plasma BUN (A) and CRE (B) levels, the weight change (C) and actual size of the kidney (D), the histopathological results in the kidney of nude mice bearing H292 tumors 21 days after treatment with saline (E), DDP (F), HDDP (G), and EHDDP (H), original magnification, ×200. The data are shown as mean±SD, *P<0.05, **P<0.01.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

Prior to describing the various embodiments, the following definitions are provided and should be used unless otherwise indicated.

“Subject” refers any animal, preferably a human patient, livestock, rodent, monkey or domestic pet.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the terms “treat” and “treating” are not limited to the case where the subject (e.g., patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

Sequence “identity” refers to the number of exactly matching residues (expressed as a percentage) in a sequence alignment between two sequences of the alignment. As used herein, percentage identity of an alignment is calculated using the number of identical positions divided by the greater of the shortest sequence or the number of equivalent positions excluding overhangs wherein internal gaps are counted as an equivalent position. For example the polypeptides GGGGGG and GGGGT have a sequence identity of 4 out of 5 or 80%. For example, the polypeptides GGGPPP and GGGAPPP have a sequence identity of 6 out of 7 or 85%.

Percent “similarity” is used to quantify the similarity between two sequences of the alignment. This method is identical to determining the identity except that certain amino acids do not have to be identical to have a match. Amino acids are classified as matches if they are among a group with similar properties according to the following amino acid groups: Aromatic—F Y W; hydrophobic—A V I L; Charged positive: R K H; Charged negative—D E; Polar—S T N Q.

### Targeted Delivery of Cisplatin Using Heparin-Cisplatin Nanoparticles

A EGFR-targeted nanoparticle system was developed based on heparin for cis-diammineplatinum(II) delivery. The formation of nanoparticles was demonstrated by chemical and biological assessments both in vitro and in vivo. There are many advantages of this nanoparticle: (1) the nanoparticle backbone, heparin, is biocompatible, biodegradable, and water-soluble, and this chemically modified heparin did not show any anticoagulant activity; (2) the relatively small size and low molecular weight ScFvEGFR is easy to obtain at lower cost, and its binding specificity to EGFR has been demonstrated; (3) the covalent attachment of ScFvEGFR to nanoparticles produced a conjugate that could be internalized into EGFR-positive cells; (4) DDP was conjugated to the nanoparticles via the exchange reaction between a —COOH group in the nanoparticle and chloride ions in DDP and was able to be released from the nanoparticles. In addition, the Pt concentration can be quantified by inductively coupled plasma mass spectrometry (ICP-MS), which makes it possible to measure the exact Pt concentration in samples (cells, blood, DNA, and organs). This assay possesses many advantages, which include (1) high sensitivity, levels as low as 2 ppb Pt can be measured; (2) high specificity due to the absence of background Pt in samples; and (3) consistency and reproducibility.

Studies of the biodistribution of targeted nanoparticles have yielded controversial results regarding the accumulation and internalization of nanoparticles in tumors. Some studies have suggested that the targeted nanoparticles did not accumulate to higher concentrations in tumor sites but could be internalized more by tumor cells, while others demonstrated that the tumor-targeted nanoparticles did selectively accumulate in the tumor mass at significantly higher concentrations compared with non-targeted nanoparticles. These discrepancies may be explained by the different nanoparticles and ligands used. Because it was expected that the tumor-targeted DDP-loaded nanoparticles could be delivered to the tumor sites quickly and be further taken up by tumor cells, so that the drug would be released intracellularly but not extracellularly, avoiding the unnecessary accumulation of nanoparticles in the body, the surface of HDDP and EHDDP nanoparticles with were not modify PEG. However, both the EHDDP and HDDP nanoparticles led to increased Pt concentrations in tumor sites compared with the free DDP, while there were significant differences between EHDDP and HDDP, and HDDP had less effect on the tumor growth rate compared with free DDP.

Although it is not intended that certain embodiments of this disclosure be limited by any particular mechanism, it is postulated that the majority of the nontargeted HDDP nanoparticles were located extracellularly rather than being internalized by the tumor cells, while only the EGFR-targeted EHDDP nanoparticles were substantially taken up by tumor cells, enabling the Pt to target the nucleus to form Pt-DNA adducts. The first step involved in nanoparticles reaching the tumor site depends mainly on their blood circulation time and the EPR effect, while active targeting only occurs after the passive accumulation of nanoparticle in tumor sites. The longer the circulation time, the more opportunity for the nanoparticles to find leaky sites in the blood vessels; therefore, the HDDP and EHDDP nanoparticles could accumulate in the tumor at relatively high concentrations due to their longer blood circulation time than free DDP. Once the nontargeted nanoparticles extravasate, they would huddle around the leaky sites of tumor blood vessels, further inhibiting the continuous accumulation of nanoparticle in tumor sites, and so the majority of the nanoparticles would be located in the tumor extracellular fluid but not inside the tumor cells. In contrast, the EGFR-targeted nanoparticles are internalized by the tumor cells via the EGFR-mediated pathway, allowing the greater accumulation of nanoparticles in the tumor.

Little is known about how the DDP enters tumor cells and forms Pt-DNA adducts; however, both passive diffusion and active transport are speculated to be involved. It is accepted that DDP is inactive when dissolved in 0.9% saline due to the relatively high concentration of chloride ions, and that once it has entered cells, it is activated because of the relatively lower concentration of chloride ions (3-20 mM) and is then available to form Pt-DNA adducts. However, one study has shown that there are no significant differences in antitumor effect between saline- and water-dissolved DDP.

In this study, cis-diammineplatinum(II) was loaded into the heparin nanoparticles by the exchange reaction between the —COOH in the heparin and chloride ions in the DDP, the loading efficacy was about 30%, and the nanoparticles were dissolved in distillated water. The DDP may be reactivated in an environment with higher chloride ion concentration (100 mM), and this was supported by our observation of the sustained release of Pt in PBS (50% released at 72 h). Since the blood half-time is only 12 min for EHDDP and 15 min for HDDP nanoparticles, the nanoparticles would be still intact before entering the tumor site, thus avoiding premature drug release. The heparin backbone of the nanoparticles may be biodegraded within the tumor cells, allowing Pt to be released in the cells.

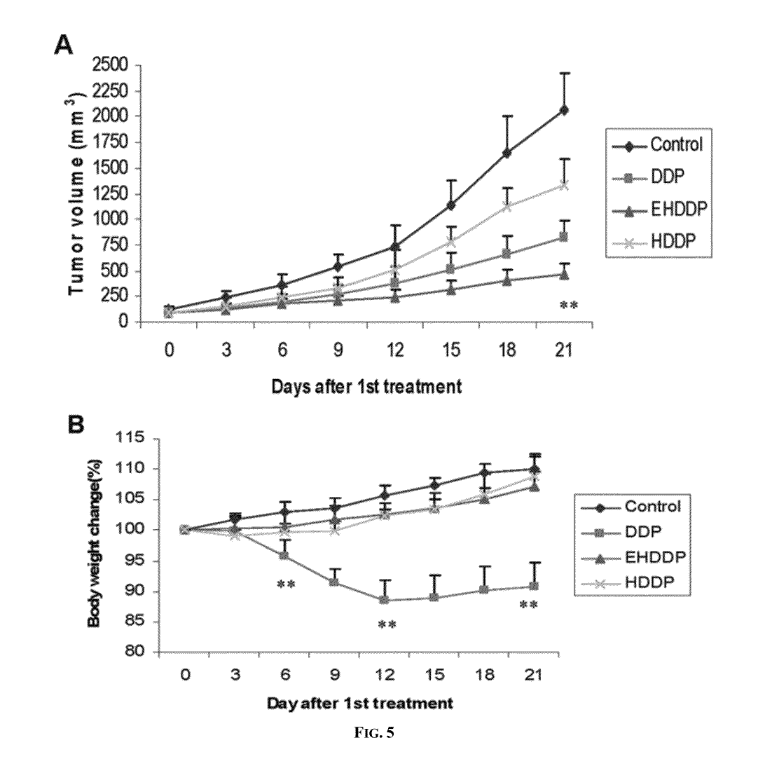

The targeted delivery of DDP by EHDDP nanoparticles significantly reduced related toxicity to the kidney and spleen. The accumulation of cis-diammineplatinum(II)-loaded nanoparticles in organs (liver, spleen, and kidney) and the tumor may be affected by different factors. The accumulation of EHDDP in tumor sites depends mainly on active targeting, as the specific binding may increase the nanoparticle retention in the tumor sites, while free DDP can be eliminated by the renal system very quickly (<5 min). There are many factors involved in the distribution of nanoparticles and their toxicity in vivo, including size, surface charge, blood circulation time, drug release profile, and metabolism process. The liver and spleen uptake of targeted and nontargeted nanoparticles play important roles in their biodistribution, and the drug-loaded nanoparticles showed a significantly increased accumulation of DDP in both liver and spleen. The nanoparticles were intact and may be captured by macrophages. Renal toxicity is considered to depend on the peak urinary DDP concentration and on the maximum DDP level in the uriniferous tubules. Studies herein showed that the retention of Pt in the kidney delivered by HDDP, EHDDP, and DDP was significantly different. Since ICP-MS can only measure the concentration of Pt but not that of the intact HDDP and EHDDP nanoparticles, it was speculated that the higher concentration of Pt in HDDP- and EHDDP-treated mice resulted mainly from inactivated HDDP and EHDDP nanoparticles. As shown in FIG. 4, the significant decrease in Pt accumulation in the kidney following free DDP treatment occurs within 4 h (65.1% decrease), while HDDP and EHDDP showed decreases of 42.7 and 33.15%, respectively, in kidney Pt levels. HDDP and EHDDP may circulate to the blood and be caught mainly by the liver and spleen again rather than being eliminated quickly by the kidneys, thus the slow and sustained release of Pt from nanoparticles significantly reduced toxicity.

Tumor-targeted heparin nanoparticles for DDP delivery to lung cancer cells was have developed. The EGFR-targeted nanoparticles led to significantly higher accumulation of cis-diammineplatinum(II) in tumor cells and enhanced the antitumor effect both in vitro and in vivo, while significantly reducing the toxicity of DDP to the spleen and kidney.

### Methods of Treating or Preventing Cancer

In certain embodiments, the disclosure relates to methods of treating cancer comprising administering an effective amount of a platinum particle disclosed herein to a subject in need thereof. In certain embodiments, the cancer is selected from head and neck squamous cell carcinoma, ovarian, and non-small cell lung cancers. In certain embodiments, the platinum particle is administered in combination with a second anti-cancer agent.

In certain embodiments, the cancer is selected from glioblastoma (GBM), breast, pancreatic, colon, metastatic lung cancers, bladder cancer, lung cancer, breast cancer, melanoma, colon and rectal cancer, non-hodgkin lymphoma, endometrial cancer, pancreatic cancer, kidney cancer, prostate cancer, leukemia, thyroid cancer, and brain cancer.

In certain embodiments, the second anti-cancer agent is temozolamide, bevacizumab, procarbazine, lomustine, vincristine, gefitinib, erlotinib, docetaxel, cis-platin, 5-fluorouracil, gemcitabine, tegafur, raltitrexed, methotrexate, cytosine arabinoside, hydroxyurea, adriamycin, bleomycin, doxorubicin, daunomycin, epirubicin, idarubicin, mitomycin-C, dactinomycin and mithramycin, vinblastine, vindesine, vinorelbine, taxol, taxotere, etoposide, teniposide, amsacrine, topotecan, camptothecin, bortezomib, anagrelide, tamoxifen, toremifene, raloxifene, droloxifene, iodoxyfene, fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab, cetuximab, dasatinib, imatinib, combretastatin, thalidomide, and/or lenalidomide or combinations thereof.

### Pharmaceutical Compositions

Generally, for pharmaceutical use, the compositions with the particle conjugates may be formulated as a pharmaceutical preparation comprising particle conjugates and a pharmaceutically acceptable carrier, diluent or excipient and/or adjuvant, and optionally one or more further pharmaceutically active compositions.

In certain embodiments, the solution comprises components selected from sodium, potassium, chloride, glucose, lactate, calcium, gelatin, succinylated gelatin, hydroxyethyl starch, dextrose, e.g., from sodium chloride, sodium lactate, calcium chloride, and potassium chloride.

In certain embodiments, the formulation is a phosphate or carbonate buffer solution optionally containing one or more saccharides.

The pharmaceutical preparations of the disclosure are typically in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, e.g. about 10, 25, 50, 100, 200, 300 or 400 mg per unit dosage.

The compositions can be administered by a variety of routes including the oral, ocular, rectal, transdermal, subcutaneous, intravenous, intramuscular or intranasal routes, depending mainly on the specific preparation used. In certain embodiments, the disclosure contemplates intravenously-delivery of an aqueous saline buffer.

The embodiments will generally be administered in an “effective amount”, by which is meant any amount of a composition that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is again made to U.S. Pat. No. 6,372,778, U.S. Pat. No. 6,369,086, U.S. Pat. No. 6,369,087 and U.S. Pat. No. 6,372,733 and the further references mentioned above, as well as to the standard handbooks, such as the latest edition of Remington's Pharmaceutical Sciences.

For an oral administration form, the composition can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, the compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compositions of the disclosure or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compositions, if desired with the substances customary therefore such as solubilizers, emulsifiers or further auxiliaries are brought into solution, suspension, or emulsion. The compositions can also be lyophilized and the lyophilizates obtained used, for example, for the production of injection or infusion preparations. Suitable solvents are, for example, water, physiological saline solution or alcohols, e.g. ethanol, propanol, glycerol, sugar solutions such as glucose or mannitol solutions, or mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

In certain embodiments, it is contemplated that these compositions can be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethyl methacrylate)); carboxypolymethylene (for example Carbopol™); carbomer; polyvinyl pyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxy methylcellulose (HMC), hydroxyethylcellulose (HEC), hydroxypropylcellulose (HPC), methylcellulose (MC), ethylcellulose (EC), carboxyethylcellulose (CEC), ethylhydroxy ethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na CMC); as well as copolymers and/or (simple) mixtures of any of the above polymers. Certain of the above-mentioned polymers may further be crosslinked by way of standard techniques.

The choice of polymer will be determined by the nature of the active ingredient/drug that is employed in the composition of the disclosure as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxy groups and hydroxypropoxyl groups will give rise to changes in the rate of release of drug from the composition. In this respect, and as stated above, it may be desirable to provide compositions of the disclosure in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release delivery systems. Particle conjugates can be entrapped in the poly(lactide-co-glycolide) microsphere depot by a number of methods, including formation of a water-in-oil emulsion with water and organic solvent (emulsion method), formation of a solid-in-oil suspension with particle dispersed in a solvent-based polymer solution (suspension method), or by dissolving the particle in a solvent-based polymer solution (dissolution method). One can attach poly(ethylene glycol) to particles (pegylation) to increase the in vivo half-life.

### EXPERIMENTAL

### Purification of ScFvEGFR from Escherichia coli Cells

Recombinant ScFvEGFR protein with a molecular weight of 25 kDa was purified from the bacterial lysates of scFv B10 transformed TG1 competent cells using Ni-NTA-agarose column separation under native conditions (Qiagen, Valencia, Calif.) as described in Yang et al., Small, 2009, 5, 235-243. A single-chain Fv (scFv) fragment contains antibody heavy- and light-chain variable domains connected with a flexible peptide linker

A typical variable heavy-chain domain is SEQ ID NO: 1
EVQLVQSGAEVKKPGSSVKVSCKASGGTFSSYAISWVRQAPGQGLEWMGGIIPIFGTANYA
QKFQGRVTITADESTSTAYMELSSLRSEDTAVYYCAREEGPYCSSTSCYGAFDIWGQGTLV
TVSS.
See Zhou et al., J. Mol. Biol. 2007, 371, 934-947.
A typical variable light-chain domain is SEQ ID NO: 2
QSVLTQDPAVSVALGQTVKITCQGDSLRSYFASWYQQKPGQAPTLVMYARNDRPAGVP DR
FSGSKSGTSASLAISGLQSEDEADYYCAAWDDSLNGYLFGAGTKLTVL.
See Zhou et al., J. Mol. Biol. 2007, 371, 934-947.

### Preparation and Characterization of Heparin-DDP (HDDP) and ScFvEGFR-Heparin-DDP (EHDDP) Nanoparticles

To further increase the DDP loading capacity and reduce the undesirable anticoagulant property of heparin, which could potentially lead to internal bleeding, the heparin was modified (Mw=12 000) with succinic anhydride according to the previously reported procedure. The succinyl ester content is 20 wt % of the resulting modified heparin (Heparin-Su), which was quantified by proton NMR with the presence of a predetermined amount of pyridine as an internal standard. For the preparation of HDDP, Heparin-Su (10 mg) and DDP (10 mg) were mixed in distilled water (10 mL) under gentle stirring at room temperature for 72 h in the dark. The heterogeneous solutions slowly became homogeneous over a period of 24 h. The free DDP was removed by dialyses against distilled water (Spectroa/Por6, MWCO=6-8 K) for 24 h. The control experiment, in which saturated free DDP in aqueous solution was dialyzed under the same conditions, showed that less than 0.5% DDP was retained in the dialysis bag. To prepare EHDDP, Heparin-Su (10 mg) and DDP (10 mg) were mixed in distilled water (10 mL) under gentle stirring at room temperature for 72 h in the dark. The resulting HDDP nanoparticles were negatively charged and covered by hydrophilic functional groups such as —COOH and —HSO3—. N-(3-Dimethylaminopropyl)-N′-ethylcarbodiimide hydrochloride (EDAC) (1 mg) and N-hydroxysuccinimide (NHS) (1 mg) were added into HDDP nanoparticle solution containing Heparin-Su (10 mg) and DDP (3.0 mg) (determined by ICP-MS) to activate carboxylic acid groups for 30 min, then ScFvEGFR (0.2 mg) was added. The mixture was stirred at room temperature for 2 h, then at 4° C. for another 48 h. The free DDP and other impurities such as EDAC and NHS were removed by dialysis as mentioned above. ScFvEGFR labeled with the fluorescent dye Oregon green 488 (Invitrogen) was used under the same conditions to determine the reaction yield. The resulting mixture was placed in a NANOSEP filter device (MWCO 100 K, Pall Life Science). After centrifugation, the fluorescence of the filtrate was measured by fluorometer (Fluoromax-2, Jobin Yvon-Spex, Horiba Group).

### Formulation and Characterization of HDDP and EHDDP Nanoparticles

To demonstrate that DDP and heparin are able to assemble into nanoparticles through coordination between the carboxyl groups and Pt²⁺, heparin and DDP were mixed in distilled water under gentle stirring. Dynamic light scattering (DLS) measurement was used to follow the formation of nanoparticles. Narrow dispersed nanoparticles were formed with an average size around 20±5 nm after 24 h as observed by DLS. The results suggest that the heparin-DDP complex forms due to the substitution of two chlorides of the DDP by the carboxyl group of the heparin.

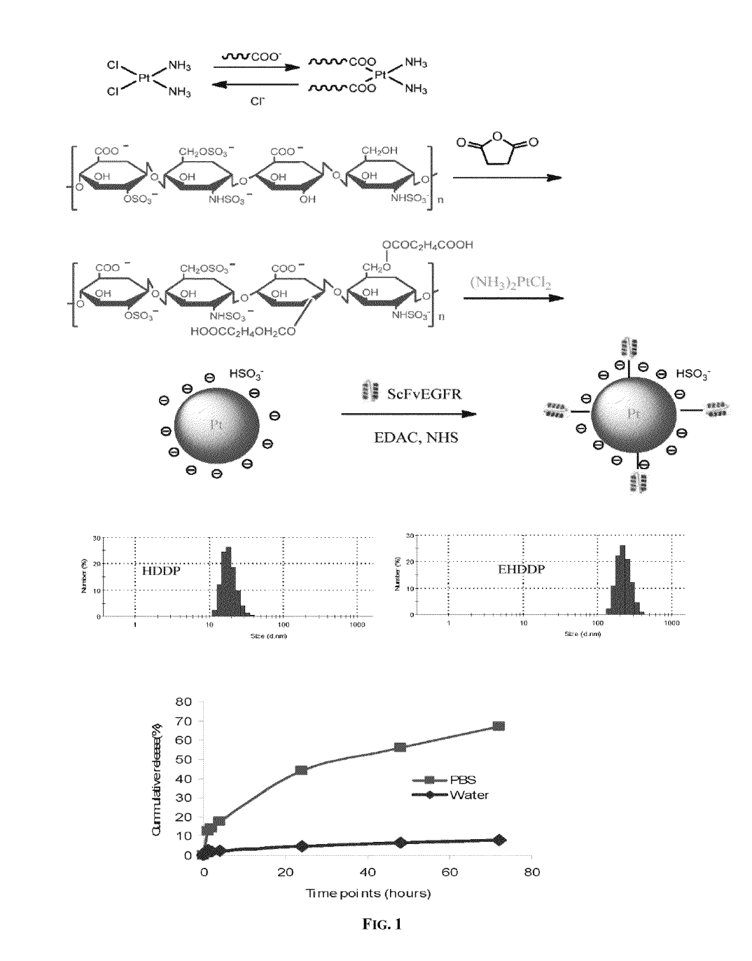

To generate EHDDP, ScFvEGFR was chemically conjugated onto the surface of the HDDP nanoparticle in the presence of EDAC and NHS (FIG. 1A). The final concentration of Pt in HDDP and EHDDP nanoparticles was about 0.20±0.03 mg/mL, as detected by ICP-MS. On the basis of the ICP-MS results, 30% of the DDP was loaded into the EHDDP nanoparticles, which demonstrated higher loading capability, and the molar ratio of ScFvEGFR/heparin/DDP was about 0.8:100:30. The DLS showed that the size of HDDP was 20±5 nm, while that of EHDDP was 150±10 nm. There are two possible reasons for the size change: (1) since the size of the conjugates is measured by dynamic light scattering, the light scatterings properties may change after the conjugation of ScFvEGFR, which leads to large DLS size; and (2) in order to conjugate ScFvEGFR onto the surface of heparin-cisplatin nanoparticles, EDAC and NHS were used as catalysts. It could potentially cause the further chemical reactions between COOH and OH group on the surface of HDDP nanoparticle, which led to the size increase. Both nanoparticles had a surface charge of about −5 mV.

As shown in FIG. 1B, 50% of the DDP was released within 72 h in PBS, suggesting sustained drug release. This led us to hypothesize that the HDDP and EHDDP are reactivated by exchanging the —COOH with the chloride in PBS. However, since the in vitro conditions are different from those in tumor cells, the mechanism of drug release from the nanoparticles in vivo was studied.

### EHDDP Nanoparticles Can Increase Pt Accumulation and Inhibit the Proliferation of Tumor Cells

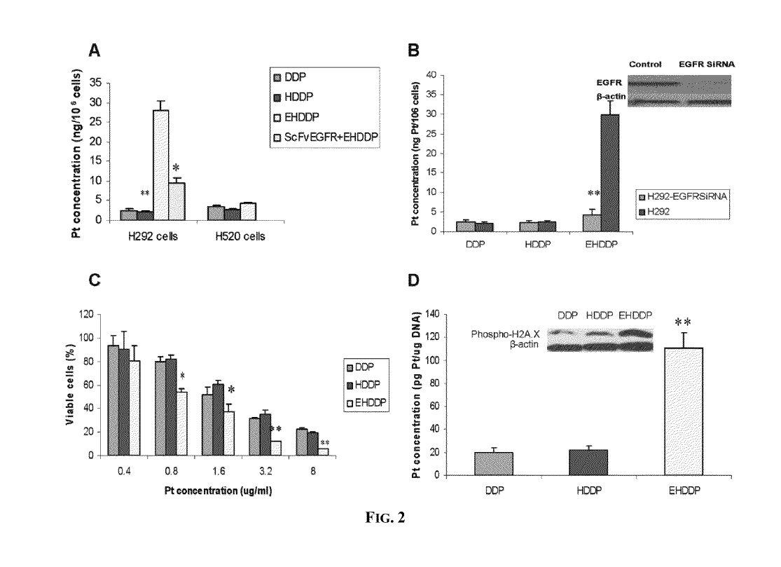

The intracellular concentration of Pt may be related to the cytotoxicity of DDP. To determine whether EHDDP nanoparticles could deliver more Pt to EGFR-positive H292 cells, ICP-MS was used to quantify the intracellular concentration of Pt. FIG. 2A shows that the intracellular Pt level was significantly higher in EHDDP-treated cells (27.91±2.45 ng Pt/10⁶ cells) than in cells treated with free DDP (2.49±0.39 ng Pt/10⁶ cells, P=0.004) or HDDP (2.14±0.20 ng Pt/10⁶ cells, P=0.003) after 24 h, while the internalization of Pt in free DDP-treated cells was not significantly different than that in cells treated with nontargeted DDP nanoparticles (P=0.25). It is thought that free DDP might enter cells by free diffusion, while EHDDP nanoparticles rapidly bind to the EGFR on the surface of H292 cells, and the EGFR-EHDDP complex is then internalized into the cells via an EGFR-mediated pathway. This was supported by a competition experiment, which showed that preincubation of H292 cells with free ScFvEGFR inhibited the uptake of Pt in H292 cells treated with EHDDP nanoparticles from 27.91±2.45 to 9.40±1.48 ng Pt/10⁶ cells (P=0.011). In addition, EGFR-negative NSCLC H520 cells showed only a limited increase in Pt accumulation when treated with EHDDP nanoparticles (4.18±0.29 ng Pt/10⁶ cells) compared with free DDP (3.38±0.42 ng Pt/10⁶ cells) (P=0.28), further demonstrating that the internalization of EHDDP nanoparticles was EGFR-dependent. To further explore the targeting specificity of EHDDP nanoparticles in vitro, EGFR siRNA was used to knockdown EGFR expression in H292 cells. EGFR expression was inhibited by about 90%, and the level of Pt internalization in EHDDP-treated H292/EGFR-siRNA cells was significantly decreased (4.25±0.85 ng Pt/10⁶cells) compared with that in H292/scrambled control siRNA cells (29.86±3.62 ng Pt/10⁶ cells) after treatment with EHDDP for 24 h (FIG. 2B).

An SRB assay was used to measure the cytotoxicity of free DDP, HDDP, and EHDDP in H292 cells at 96 h after treatment. The IC50 of EHDDP (Pt concentration=1.08±0.12 μg/mL) in H292 cells was significantly lower than that of DDP (2.12±0.20 μg/mL, P=0.03) and HDDP (2.33±0.22 μg/mL, P=0.01). As shown in FIG. 2C, the inhibition of cell proliferation by EHDDP was significantly greater than that of free DDP and nontargeted HDDP nanoparticles.

### EHDDP Nanoparticles Increase the Formation of Intracellular Pt-DNA Adducts

The increase in intracellular Pt concentration seen in H292 cells treated with EHDDP nanoparticles, measured by ICP-MS, reflects the enhanced internalization of the total DDP loaded in the nanoparticles, not only the DDP activated from the nanoparticles, yet only this activated DDP can enter the nucleus and form Pt-DNA adducts. To quantify the levels of bioavailable DDP in the internalized EHDDP nanoparticles, H292 cells were incubated with free DDP, EGFR-targeted or nontargeted DDP nanoparticles at 37° C. for 24 h, followed by purifying the total DNA of treated cells and measuring the Pt concentration of DNA using ICP-MS. Data showed that the Pt level associated with cellular DNA was significantly greater in EHDDP-treated cells (110.93±12.89 pg Pt/μg DNA) than in cells treated with DDP (19.68±4.35 pg Pt/μg DNA, P=0.003) or HDDP (22.17±3.37 pg Pt/μg DNA, P=0.002), while free DDP did not show any significant difference in DNA-associated Pt levels than HDDP (P=0.2) (FIG. 2D). Phosphorylation of H2A.X can act as a highly sensitive and general marker of DNA damage induced by DDP; therefore, the phosphorylation of H2A.X was assessed in H292 cells treated with DDP, HDDP, and EHDDP nanoparticle (0.4 μg/mL) for 24 h. The expression level of phosphorylated H2A.X was significantly higher in EHDDP-treated cells than in DDP- or HDDP-treated cells (FIG. 2D), which was consistent with the level of Pt-DNA adduct formation detected by ICP-MS.

### EHDDP Nanoparticles Did Not Show Any Anticoagulant Activity

Heparin exerts its anticoagulant activity by reversibly binding to antithrombin III. The structure of heparin was modified by using succinic anhydride to inactivate its anticoagulant function. The anticoagulant activities of EHDDP were determined by FXa-dependent coagulant assay. Data showed that the EHDDP nanoparticles had undetectable anticoagulant activity, while that of unmodified heparin was 178 U/mg.

### EHDDP Nanoparticles Prolonged Blood Circulation Time and Changed the Biodistribution of Pt In Vivo

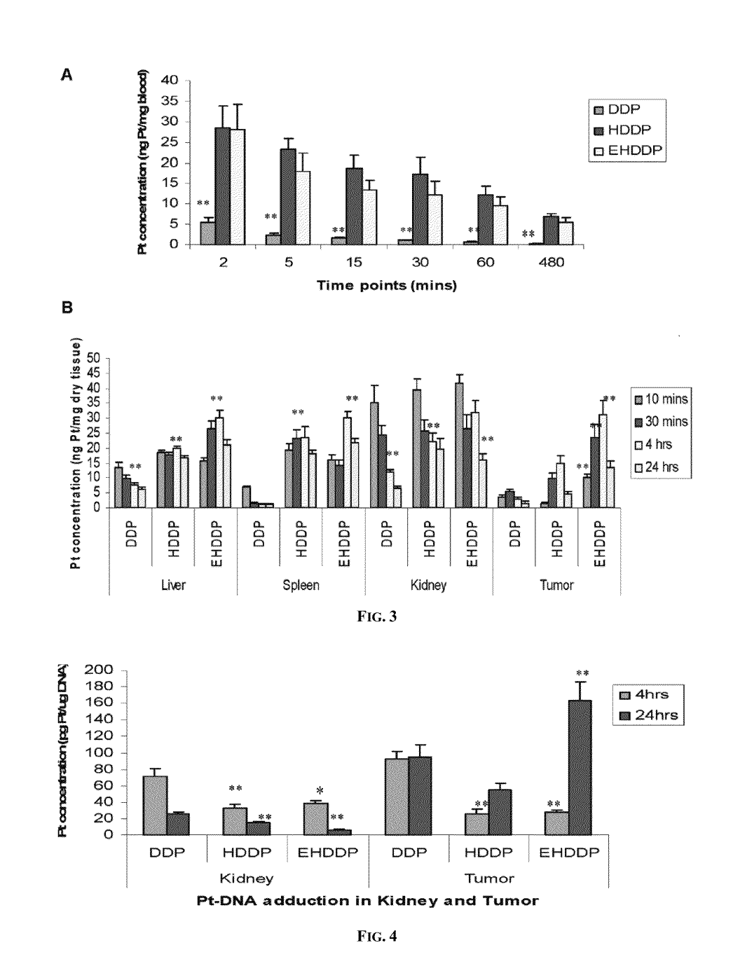

The plasma Pt level was measured by ICP-MS after intravenous injection of free DDP, HDDP, and EHDDP nanoparticles to determine the blood half-times of these three drugs as 2, 12, and 15 min, respectively. FIG. 3A shows that the HDDP and EHDDP nanoparticles significantly increased the circulation time of Pt: 30 min after systemic administration, the Pt concentration in mice treated with free DDP was 1.17±0.12 ng/mg blood compared with 12.31±3.2 ng/mg in mice treated with EHDDP (P=0.003). After 8 h, the Pt concentration further decreased in DDP-treated mice (0.36±0.09 ng/mg blood), while mice treated with EHDDP nanoparticles maintained a relatively higher Pt level (5.61±1.22 ng/mg blood, P=0.006). The results also showed that the ScFvEGFR on the surface of heparin nanoparticles had significant effects on the Pt concentration (12.31±3.2 ng/mg blood) in blood compared with the HDDP (17.55±0.48 ng/mg blood, P=0.016) 30 min after iv injection, while no significant differences were found after 60 min (P=0.07).

To study the effects of the HDDP and EHDDP nanoparticles on the biodistribution of Pt in vivo, the main organs were harvested including the liver, spleen, and kidney at different time points (10 min, 30 min, 4 h, and 24 h) after the mice were injected intravenously with a single dose of free DDP and DDP-loaded nanoparticles (Pt 2.5 mg/kg) and the Pt concentrations were quantified in the tissue samples using ICP-MS. FIG. 3B shows that significantly greater Pt accumulation was seen in the liver and spleen of HDDP and EHDDP nanoparticle-treated mice than in animals treated with free DDP, indicating that HDDP and EHDDP nanoparticles were taken up by the reticuloendothelial system (RES) after their systemic administration. In contrast, most of the free Pt is eliminated through glomerular filtration. The accumulation of Pt in the liver 4 h following HDDP and EHDDP delivery was 2.7-fold (20.81±2.59 ng/mg dry tissue) and 4-fold (30.88±5.07 ng/mg dry tissue), respectively, greater than that of free DDP (7.71±1.61 ng/mg dry tissue) (P=0.0027 and 0.0018, respectively). The accumulation of Pt in the spleen 4 h following HDDP and EHDDP delivery was 18.6-fold (23.51±3.56 ng/mg dry tissue) and 22.1-fold (28.16±5.68 ng/mg dry tissue), respectively, greater than that of free DDP (1.27±0.19 ng/mg dry tissue) (P=0.008 and 0.0017, respectively). It should be noted that the changes in biodistribution of Pt in the liver and spleen delivered by free DDP, HDDP, and EHDDP were significantly different during the first 4 h after administration. The free DDP-induced Pt accumulation in the liver and spleen significantly decreased from 10 min to 4 h (42.5%, P=0.033, 82.86%, P=0.0015, respectively), while EHDDP induced significantly increased Pt accumulation between 10 min and 4 h in the liver and spleen (90.2%, P=0.0067, 88.9%, P=0.022, respectively). After 24 h, the Pt concentration in the liver significantly decreased in both DDP-treated mice (20% decrease compared with 4 h, P=0.007) and EHDDP-treated mice (30% decrease, P=0.0036), and the Pt concentration in the spleen also significantly decreased in HDDP-treated (23.2% decrease, P=0.042) and EHDDP-treated (28.1% decrease, P=0.034) groups. It is believed that the majority of the HDDP and EHDDP nanoparticles are still intact within 4 h and are degraded gradually in vivo. There were no differences in Pt concentrations delivered by DDP, HDDP, and EHDDP in the liver, spleen, and kidney 28 days after treatment.

Since the kidney is the main organ that eliminates Pt, and the renal toxicity induced by DDP is due to damage caused by Pt to proximal tubular cells and the dilated tubular lumen, the accumulation of Pt in the kidney was investigated at different time points following delivery of free DDP, HDDP, and EHDDP nanoparticles. As shown in FIG. 3B, 10 min after systemic administration of DDP, HDDP, and EHDDP, the Pt concentrations in mice kidney were 35.25±5.70, 39.48±3.52 (P=0.079), and 41.74±2.93 (P=0.18) ng/mg dry tissue, respectively. The Pt concentrations decreased in the kidney over time; after 4 h, the HDDP and EHDDP nanoparticle-treated groups showed significantly higher Pt concentrations (22.1.6±2.82 and 31.7±4.03 ng/mg dry tissue) than free DDP (11.9±0.79 ng/mg dry tissue) (P=0.013 and 0.019, respectively), and after 24 h, the HDDP and EHDDP nanoparticle-treated groups still showed significantly higher Pt concentrations (19.6±3.44 and 16.09±1.96 ng/mg dry tissue) than in the free DDP group (6.39±0.67 ng/mg dry tissue) (P=0.014 and 0.024, respectively).

One of the main advantages of using targeted nanoparticles for drug delivery is that they can selectively accumulate in the tumor sites. Tumor tissues were collected at different time points (10 min, 30 min, 4 h, and 24 h) after iv injection of DDP, HDDP, and EHDDP nanoparticles and measured the total Pt concentrations. As FIG. 3B shows, after 10 min, the tumor Pt levels were significantly higher in mice treated with EHDDP nanoparticles (10.01±1.21 ng/mg dry tissue) than with free DDP (3.58±0.91 ng/mg dry tissue, P=0.034) or HDDP (1.55±0.18 ng/mg dry tissue, P=0.004), while there was no significant difference between the DDP and HDDP groups (P=0.084). After 4 h, the tumor levels of Pt increased in mice treated with HDDP and EHDDP nanoparticles but decreased in mice treated with DDP; furthermore, the targeted EHDDP nanoparticles induced greater Pt tumor accumulation (31.3±4.7 ng/mg dry tissue) than the nontargeted HDDP nanoparticles (14.68±2.52 ng/mg dry tissue, P=0.0056), and HDDP delivered significantly higher levels of Pt to the tumor mass than free DDP (P=0.008). After 24 h, the Pt levels in tumors of all groups decreased; however, the accumulation of Pt in the EHDDP group (13.45±2.15 ng/mg dry tissue) was still significantly higher than that in the HDDP group (4.87±0.67 ng/mg dry tissue, P=0.026) or the DDP group (1.48±0.52 ng/mg dry tissue, P=0.02).

Since only the activated Pt can form intracellular Pt-DNA adducts, DNA was purified from the kidney at 4 and 24 h after treatment and quantified the Pt level in the total DNA. As shown in FIG. 4, the Pt concentrations after 4 and 24 h were significantly higher in DNA purified from mice kidney treated with DDP (71.78±9.02 and 25.72±2.78 pg/μg DNA, respectively) than those treated with HDDP (32.91±5.07 and 14.88±1.71 pg/μg DNA, P=0.003 and 0.0039, respectively) or EHDDP (38.61±6.01 and 5.87±1.30 pg/μg DNA, P=0.042 and 0.001, respectively). It is believed that most of the Pt in HDDP and EHDDP nanoparticles was not released after 24 h in vivo but will release gradually. The level of Pt in Pt-DNA adducts was quantified in the NSCLC xenograft tumors at 4 and 24 h after treatment with DDP, HDDP, and EHDDP nanoparticles (single dose iv injection, Pt 2.5 mg/kg), and results (FIG. 4) showed that the formation of Pt-DNA adducts in DDP-treated tumors (92.21±8.96 pg/μg DNA) was significantly greater than that in HDDP-treated (26.09±5.15 pg/μg DNA, P=0.0029) and EHDDP-treated (28.21±2.06 pg/μg DNA, P=0.0038) groups after 4 h. After 24 h, the EHDDP nanoparticle-treated tumors showed significantly increased formation of Pt-DNA adducts (163.12±26.59 pg/μg DNA) than that of DDP-treated (94.68±15.01 pg/μg DNA, P=0.0039) and HDDP-treated (55.01±7.72 pg/μg DNA, P=0.0061) tumors.

### EHDDP Nanoparticles Enhance Antitumor Effects While Significantly Reducing the Toxicity to Kidney and Spleen In Vivo

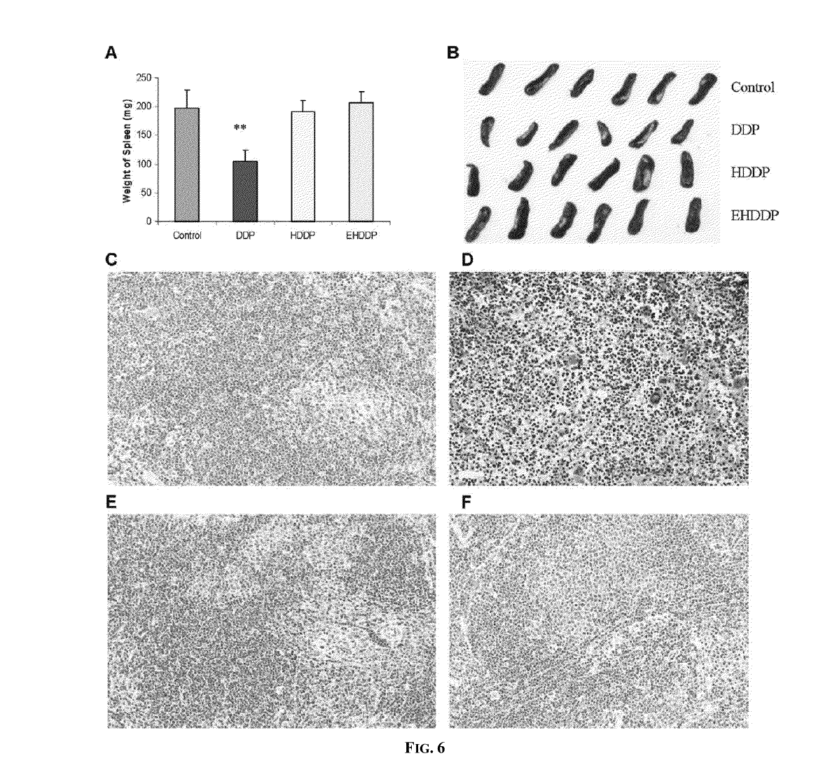

Nude mice bearing human NSCLC H292 tumors received treatment with free DDP, HDDP, and EHDDP nanoparticles (Pt 2.5 mg/kg, 5 iv doses, 3 day intervals). EHDDP nanoparticles showed a significant decrease in the tumor growth rate as compared with the HDDP (P<0.01) and free DDP (P<0.05) groups, while the nontargeted HDDP nanoparticles did not enhance the antitumor effects of DDP (FIG. 5A). Although the HDDP nanoparticles carried more Pt into tumor site than the free DDP, the level of bioavailable Pt was postulated to be less than with free DDP. Although the majority of the nontargeted HDDP nanoparticles will diffuse away from the tumor site, some nanoparticles can enter the tumor cells by passive diffusion (EPR effect) and release a relatively lower concentration of Pt, which may have some effect on tumor growth.

Mice receiving the free DDP showed a significant loss of body weight, with about 5% weight loss by the sixth day (P=0.002) and 12.7% by the 12th day (P<0.001) after treatment. In contrast, mice treated with HDDP and EHDDP nanoparticles did not show any significant weight loss compared with the control group which received saline injection (FIG. 5B).

All the mice were sacrificed after 21 days of treatment since the tumor volumes in the saline control group had reached 2000 cm3. The mice blood, liver, spleen, and kidney were collected. A significant shrinkage of the spleen was found in mice treated with free DDP; the spleen weight (104.94±21.92 mg) was 47.2% lower than that of the control group (197.83±31.05 mg, P=0.0005), while spleen weights in the HDDP (190.72±19.62 mg) and EHDDP (206.82±35.89 mg) groups did not show any significant change (P=0.35 and 0.66, respectively) (FIGS. 6A,B). Damage to the spleen was further confirmed by histopathological changes, which showed a marked hypocellularity in the white pulp and aggregation of neutrophils in the red pulp with minor necrosis in mice treated with DDP but not HDDP or EHDDP (FIG. 6C-F).

Serum glutamic pyruvate transaminase (GPT) and glutamic oxaloacetic transaminase (GOT) levels were quantified to evaluate mice hepatic function. The GPT levels did not change significantly in any of the treated groups (DDP=48±7.04 U/L; HDDP=41.33±11.64 U/L; EHDDP=44±9.64 U/L) compared with the control group (40.66±10.24 U/L). The GOT level in DDP-treated mice was 148.6±32.71 U/L, which was not significantly different from that in the control group (138±30.08 U/L) (P=0.35), or the HDDP (154.6±22.75 U/L) and EHDDP (144.3±28.1 U/L) groups (P=0.41 and 0.43, respectively). Consistent with this finding, histopathological studies did not show any significant changes in any of the treated groups compared with the control group under the microscope.

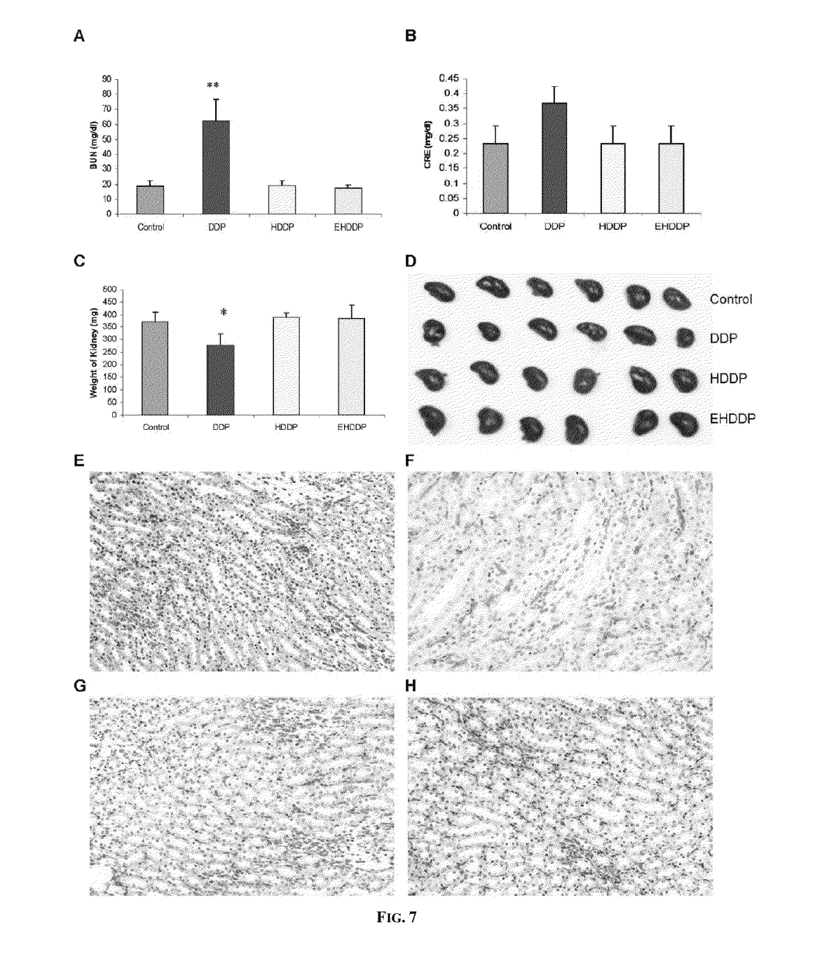

Serum blood urea nitrogen (BUN) and creatinine (CRE) levels were measured to assess renal function, and the DDP-treated mice showed a significantly higher concentration of BUN (62.67±14.37 mg/dL) than the control group (19.67±3.73 mg/dL, P=0.0076), while levels of BUN in mice treated with HDDP (19.33±3.24 mg/dL) and EHDDP (17.67±2.084 mg/dL) nanoparticles were not significantly increased compared with the control group (P=0.29 and 0.35, respectively, FIG. 7A). Serum CRE levels did show an increase in DDP-treated mice (0.36±0.05 mg/dL) compared with the control (0.233±0.057 mg/dL), although the difference was not significant, and the HDDP (0.25±0.053 mg/dL) and EHDDP (0.23±0.046 mg/dL) groups did not show any significant changes in CRE levels compared with the control group (P=0.18 and 0.39, respectively, FIG. 7B). The significantly elevated BUN in DDP-treated mice indicated damage to renal function, although the serum CRE level did not increase significantly compared with the control group, and such impaired renal excretion of urea may be due to dehydration resulting from the DDP treatment. Kidney toxicity was evidenced by the significant loss of kidney weight in the DDP-treated mice (277.21±45.27 mg), 25.56% lower than that in the control mice (372.4±37.68 mg, P=0.013), while no kidney weight loss was seen in HDDP (390.33±19.08 mg) and EHDDP (384.84±54.95 mg) groups compared with the control group (P=0.24 and 0.63, respectively), as shown in FIGS. 7C,D. Histopathology analysis showed that the structure of glomeruli in the DDP-treated group did not change noticeably, but the epithelial cells in the convoluted proximal tubule became swollen and enlarged, and some of the tubular lumen disappeared (FIG. 7F). In contrast, the HDDP (FIG. 7G) and EHDDP nanoparticles (FIG. 7H) did not induce any significant changes in kidney histopathology compared with the control group (FIG. 7E).

### Characterization of the EGFR-Targeted Heparin-DDP (EHDDP) Nanoparticles

The mean diameter of HDDP and EHDDP nanoparticles was evaluated by dynamic light scattering (DLS) measurements using a model 127-35 laser (Spectra Physics, CA) operating at 633 nm and 25° C. The zeta-potential of the nanoparticles was determined using microelectrophoresis. The loading of nanoparticles with DDP was determined using ICP-MS.

### Study of the In Vitro Release of DDP from EHDDP Nanoparticles

Release of Pt from EHDDP nanoparticles in PBS at 37° C. was evaluated as follows: 20 mL of EHDDP nanoparticle solution was placed into dialysis bags and immersed into 480 mL of PBS. At definite time intervals, 1 mL of the solution outside the dialysis bag was analyzed by ICP-MS to determine the amount of DDP released from EHDDP nanoparticles.

### Cell Culture

Two cell lines (EGFR-positive H292 and EGFR-negative H520) were selected for our study to represent NSCLC cells. Both cells were maintained in RPMI1640 medium supplemented with 10% heat-inactivated fetal bovine serum (FBS) and antibiotics (streptomycin, penicillin G, and amphotericin B) in a 37° C., 5% CO₂ humidified incubator.

### In Vitro Cytotoxicity Assay

To test the effects of free DDP, HDDP, and EHDDP nanoparticles on cell growth of tumor cells, sulforhodamine B (SRB) cytotoxicity assays were used. H292 and H520 cells maintained in medium with 2% FBS were seeded in 96-well plates at a density of 5000 cells/well overnight prior to drug treatment. DDP, HDDP, or EHDDP nanoparticles were added in various concentrations, followed by incubation at 37° C. and 5% CO₂ for 96 h. Cells were fixed for 1 h with 10% cold trichloroacetic acid. Plates were washed five times in water, air-dried, and then stained with 0.4% SRB for 10 min. After washing four times in 1% acetic acid and air-drying, bound SRB was dissolved in 10 mM Tris base (pH 10.5). Plates were read in a microplate reader by measuring absorbance at 492 nm. The percent survival was then calculated based upon the absorbance values relative to untreated samples. The experiment was repeated three times.

### Quantification of Intracellular Platinum Accumulation and DNA-Adduct Formation In Vitro

H292 cells (1×10⁶) were incubated for 24 h at 37° C. with free DDP or HDDP, EHDDP nanoparticles (0.4 μg/mL). EGFR-negative H520 cells were used as negative control. To study the internalization of DDP in the presence of free ScFvEGFR, H292 cells were incubated with different concentrations of ScFvEGFR at 37° C. for 20 min, and then EHDDP nanoparticles were added and incubated for another 24 h. To remove surface-bound DDP, cells were washed three times with ice-cold PBS, incubated with 1.5 mL of 0.15 M sodium chloride (pH 3.0 was adjusted by acetic acid) for 3 min at 4° C., then rinsed with 2 mL of cold PBS, harvested by scraping in ice-cold PBS, centrifuged, and cell numbers were counted. The cell pellet was digested in 65% (v/v) nitric acid at 75° C. for 2 h. The Pt content was analyzed by ICP-MS. Cellular Pt levels were expressed in ng Pt/10⁶ cells. To further check the targeting specificity of EHDDP nanoparticles in vitro, EGFR was knocked down by EGFR small interfering RNAs (siRNA). H292 cells were transfected with EGFR siRNA (sc-29301 from Santa Cruz Bio) (100 pmol) or scrambled siRNA (sc-37007 from Santa Cruz Bio) (100 pmol) as a nonspecific control according to the manufacturer's protocols. Briefly, in a 6-well tissue culture plate, 2×10⁵ cells per well were seeded in 2 mL of antibiotic-free RPMI 1640 medium supplemented with 10% FBS and, on the following day, transfected with siRNAs at the final concentration of 100 pmol. Cells were harvested 24 h later, whole cell lysates were extracted using lysis buffer, and 15 μg of protein was separated on 8-12% SDS-PAGE, then transferred onto a polyvinylidene difluoride membrane (Millipore Corp., Billerica, Mass.) and immunoblotted with specific antibodies against EGFR (sc-03 from Santa Cruz Bio). Mouse anti-β-actin antibody (Trevigen, Gaithersburg, Md.) was used as a sample loading control. Immunostained protein bands were detected with an enhanced chemiluminescence kit (Thermo Scientific, Rockfield, Ill.). Twenty-four hours after transfection of EGFR siRNA, cells were treated with DDP, HDDP, and EHDDP (0.4 μg/mL) for 24 h and Pt concentration was quantified by ICP-MS as described above. The data are shown as mean±SD (n=3). DDP-DNA adduct formation was measured using 1×10⁷ cells per condition. DNA was isolated by using DNA purification kit (Invitrogen, USA), and the DNA was digested with DNase I before analysis of Pt content by ICP-MS. Total DNA adducts were expressed in pg/μm DNA. In addition, the expression level of the phosphorylation of H2A.X was detected by Western Blot as described above using the Phospho-Histone H2A.X (Ser139) (20E3) rabbit mAb (Cell Signaling Technology, Inc.).

### Coagulation Assays

The anticoagulant activities of EHDDP were determined by FXa-dependent coagulant assay using Coatest Heparin (Helena Laboratories, Beaumont, Texas). Briefly, 200 μL of standard samples of heparin (concentrations of heparin from 0.01 to 0.07 unit/mL) and EHDDP were incubated at 37° C. for 3-4 min, then 100 μL of FXa (0.355 nkat) was added and mixed well. The mixture was incubated at 37° C. for 30 s, and 200 μL of 1 mM of chromogenic substrate S-2222 was added. The mixture was incubated at 37° C. for 3 min. The reaction was stopped by adding 300 μL of 20% (v/v) acetic acid. The sample was transferred to a semi-microplate, and the absorbance of the samples at 405 nm was detected. The anticoagulant activity was calculated based on the standard curve.

### Concentration Profiles of Pt in Mice Peripheral Blood

DDP, HDDP, and EHDDP (Pt concentration 2.5 mg/kg) nanoparticles were injected into nude mice by the tail vein. Blood (30 μl) was collected from the retro-orbital plexus at different time points (2, 5, 15, 30, 60, 120, 480, and 1440 min). The whole blood samples were dissolved in 65% (v/v) nitric acid at 75° C. for 2 h, and Pt concentration was measured by ICP-MS.

### Biodistribution of EHDDP Nanoparticles and Formation of Pt-DNA Adducts In Vivo

To evaluate the biodistribution of DDP, HDDP, and EHDDP nanoparticles in tumor-bearing mice, the agents were injected into mice (3 mice each group) by tail vein, and 10 min, 30 min, 4 h, and 24 h later, mice were sacrificed. Blood, organs (liver, spleen, kidney, and lung), and tumor tissue were harvested, half of the organs were dissolved in 65% (v/v) nitric acid at 75° C. for 2 h, and Pt concentrations were measured by ICP-MS. The blood and other half of the organs were used to extract DNA, and the Pt concentration in DNA was quantified as described above.

### In Vivo Antitumor Efficacy of EHDDP Nanoparticles

The animal experiments were approved by the Animal Care and Use Committee of Emory University. Twenty-four nude mice (athymic nu/nu, Taconic, NY), aged 4-6 weeks (about 20 g weight), were randomly divided into four groups. H292 tumor cells (1×10⁶ cells per mouse) were injected subcutaneously into the left flank of male nude mice. When the tumor volume was approximately 100 mm3, the mice were injected intravenously via the tail vein five times at 3 day intervals with free DDP (200 μL of aqueous DDP solution) at a dose of Pt 2.5 mg/kg or DDP-loaded nanoparticles at the same dose. The control group was administered saline. The tumor size was measured three times a week. The antitumor activity was evaluated in terms of the tumor size at different times postadministration, which was estimated by the following equation: V=6×larger diameter×(smaller diameter)2/π. Growth curves were plotted using the average tumor size within each experimental group at the set time points. The whole group of mice was sacrificed once the size of any tumor in that group reached 2 cm in diameter. To evaluate the tolerance of nude mice to EHDDP nanoparticles, the body weight and physical state of the mice were measured simultaneously as an indicator of systemic toxicity.

1. A platinum complex comprising at least one polysaccharide ligand.

2. The platinum complex of claim 1, wherein the complex is a cis-diammine platinum(II) complex.

3. The platinum complex of claim 2, wherein the polysaccharide ligand is heparin or modified heparin.

4. A platinum particle coated with a polysaccharide conjugated to a polypeptide that has affinity for a cell surface cancer marker.

5. The platinum particle of claim 4, wherein the polysaccharide is a heparin or modified heparin.

6. The platinum particle of claim 5, wherein the heparin is modified with succinic anhydride.

7. The platinum particle of claim 5, wherein the heparin is conjugated to the polypeptide that targets EGFR.

8. The platinum particle of claim 7, wherein the polypeptide is ScFvEGFR.

9. A pharmaceutical composition comprising a particle of claim 4 and a pharmaceutically acceptable excipient.

10. A method of treating cancer comprising administering an effective amount of a platinum particle of claim 4 to a subject in need thereof.

11. The method of claim 10, wherein the cancer is selected from head and neck squamous cell carcinoma, ovarian cancer, non-small cell lung cancer and other solid tumors.

12. The method of claim 10, wherein the platinum particle is administered in combination with a second anti-cancer agent.
